(19) United States
(12) Patent Application Publication
(10) Pub. No.: US 2026/0199575 A1
(43) Pub. Date: Jul. 16, 2026

(54) INLINE MANIPULATION OF CELLS AND CELL CLUSTERS

(71) Applicant: GEORGIA TECH RESEARCH CORPORATION, Atlanta, GA (US)

(72) Inventors: Ali Fatih SARIOGLU, Atlanta, GA (US); Mert BOYA, Atlanta, GA (US); Aref VALIPOUR, Atlanta, GA (US); Stephen N. HOUSLEY, Atlanta, GA (US)

(21) Appl. No.: 19/136,238

(22) PCT Filed: Dec. 7, 2023
(86) PCT No.: PCT/US2023/082980

§ 371 Date: Jun. 5, 2025

Related U.S. Application Data
(60) Provisional application No. 63/386,418, filed on Dec. 7, 2022.

(51) Int. Cl. A61M 1/34; A61F 2/01; A61M 1/36

(52) U.S. Cl. CPC A61M 1/34; A61F 2/01; A61M 1/3627; A61M 2202/0413; A61M 2202/20; A61M 2205/3306; A61M 2205/36

(57) ABSTRACT

Disclosed are methods and systems relating to removing cell clusters from blood samples.

### CROSS-REFERENCE TO RELATED APPLICATIONS

This application claims the benefit of priority to U.S. Provisional Application No. 63/386,418, filed Dec. 7, 2022, entitled “Inline Manipulation of Cells and Cell Clusters,” which is incorporated by reference herein in its entirety.

### STATEMENT OF GOVERNMENT SUPPORT

This invention was made with government support under Grant No. W81XWH-20-1-0649 (A.F.S.) awarded by the Office of the Assistant Secretary of Defense for Health Affairs. The government has certain rights in the invention.

### BACKGROUND

Cancer tumor metastasis is responsible for over 90% of all cancer-related deaths. During metastasis, single tumor cells or a group of cells (clusters) detach from a primary tumor location, where they intravasate into the bloodstream, circulate inside the body through blood, and extravasate to form a new metastatic site. Circulating tumor cell (CTC) clusters, which are typically formed by the aggregation of individual cancer cells, have garnered recent attention in cancer management due to their underlying significance in both diagnostics and clinical applications. Although relatively uncommon in the blood stream compared to individual CTCs, the metastatic propensity of CTC clusters has been shown to be up to 50 times higher than individual cells, suggesting that clinical removal of CTC clusters could be an effective way to ameliorate cancer metastasis.

Despite their known deleterious impact on cancer metastasis, inline removal of CTC clusters has not yet been fully explored. Current ex vivo enrichment technologies rely on small blood volumes that can be extracted from the patients at a single time point due to safety concerns, such as the formation of blood clots and hemolysis. Furthermore, the low exchange of blood combined with the scarcity of CTC clusters in circulating blood yields a low number of isolatable CTC clusters, which in turn makes early-stage monitoring difficult.

There is a benefit to improving detection and/or reducing metastatic risk of cancer.

### SUMMARY

In one example, the present disclosure relates to an extracorporeal circulation system designed for in-line enrichment of CTC clusters for longitudinal screening of truly large blood volumes.

In various aspects, disclosed herein is a system for the in-line removal of cell clusters from a bodily fluid (e.g., blood sample) of a subject using a high-flow filter assembly having a micro-structure filter substrate that forms a plurality of filtering elements (e.g., semipermeable microwells). The filter assembly is configured to receive (e.g., directly) an inlet fluid (e.g., unfiltered blood) from a subject at a first side and output a filtered output at a second side, wherein each filtering element defines a plurality of apertures extending between the first side and the second side of the filter, each filtering element forming a cluster-exclusion barrier (e.g., a size-exclusion barrier) in a trapping region of the filtering element to substantially prevent cell clusters of multiple cells (e.g., circulating tumor cell (CTC) clusters) from passing from the first side to the second side.

In some aspects, the system includes a pump (e.g., a centrifugal pump, peristaltic pump) configured to move the inlet fluid towards or from the high-flow filter assembly.

In some aspects, the cluster-exclusion barrier comprises an interconnected mesh defining a plurality of spaced openings.

In some aspects, the cluster-exclusion barrier comprises cluster-breaking elements configured to at least partially separate cell clusters.

In some aspects, the system further comprises a bubble remover in fluid communication with the high-flow filter assembly, wherein the bubble remover is configured to remove gases (e.g., air bubbles) from the filtered output.

In some aspects, the bubble remover comprises an inlet stream of the filtered output entering a housing of the bubble remover, wherein the inlet stream is configured to contact a substantially immiscible liquid (e.g., saline) having a lower density than the filtered output.

In some aspects, the inlet stream is configured to enter the bubble remover substantially towards a side wall of the housing.

In some aspects, the inlet stream is configured to enter the bubble remover substantially away from a side wall of the housing.

In some aspects, the system further includes a heating unit (e.g., a thermal lamp) configured to supply thermal energy to the fluid inlet and/or the filtered outlet.

In some aspects, the filter substrate of the high-flow filter assembly is coupled to a filter holder.

In some aspects, the filter holder comprises a sealing region, wherein the sealing region defines an area (e.g., a circle) enclosing at least a portion of the high-flow filter assembly, wherein the sealing region is configured to substantially prevent the inlet fluid from passing through the filter substrate outside of the area.

In some aspects, the sealing region is substantially non-porous to the inlet fluid.

In some aspects, a surface utilization rate of the filter substrate is 5% or more (e.g., 10% or more, 15% or more, 20% or more, 25% or more, 30% or more, 35% or more, 40% or more, 45% or more, or 50% or more).

In some aspects, a surface density of the apertures on the filter substrate is 500 apertures/mm² or more (1000 apertures/mm² or more, 1500 apertures/mm² or more, 2000 apertures/mm² or more, 2500 apertures/mm² or more, or 3000 apertures/mm² or more.

In some aspects, the high-flow filter assembly is substantially flat. In some examples, the high-flow filter assembly has a thickness of 1 μm to 1000 μm (e.g., from 1 μm to 500 μm, from 1 μm to 400 μm, from 1 μm to 300 μm, from 1 μm to 200 μm, from 1 μm to 100 μm, from 1 μm to 50 μm, from 1 μm to 40 μm, from 1 μm to 30 μm, from 1 μm to 20 μm, from 1 μm to 10 μm, or from 5 μm to 10 μm). In some aspects, the high-flow filter assembly has a three-dimensional shape (e.g., a conical structure).

In some aspects, the filter substrate comprises a biocompatible material. In some aspects, the filter substrate comprises a UV-curable polymer. In some aspects, the filter substrate comprises a printable material (e.g., an acrylate polymer). In some aspects, the filter substrate comprises a disposable material.

In some aspects, the system is a closed-loop system.

In some aspects, the system further includes sensors (e.g., temperature, pressure, and/or flow rate sensors).

In some aspects, the system further includes an imaging unit (e.g., a microscope) configured to image and/or screen retained cell clusters.

In some aspects, the system further includes a display.

In some aspects, the system further includes a plurality of high-flow filter assemblies arranged in parallel fluid circuits.

In some aspects, the system further includes a plurality of high-flow filter assemblies arranged in sequential fluid circuits (e.g., to sort cell clusters by size).

In some aspects, the high-flow filter assembly is operatively coupled to an implantable device.

In some aspects, the high-flow filter assembly is operatively coupled to a stent.

Also disclosed herein are methods for the in-line removal of cell clusters. In some aspects, the method includes: causing a volume of a fluid of a subject (e.g., blood) to circulate within the in-line filtration systems described herein.

In some aspects, the method further includes causing the filtered output to be returned to the subject. In some aspects, the method also includes isolating retained cell clusters from the high-flow filter assembly.

In some aspects, the cell clusters are isolated by flowing a recovery fluid through the high-flow filter assembly in a flow direction from the second side to the first side.

In some aspects, the isolated cell clusters are used to diagnose, quantify, characterize, and/or monitor a cancer in the subject. In some aspects, the cancer is selected from the group consisting of prostate cancer, ovarian cancer, breast cancer, and medulloblastoma.

In some aspects, the method further includes starting, suggesting, adjusting and/or directing a treatment parameter in response to the isolated cell clusters.

In some aspects, a volumetric flow rate of the blood is 1 mL/hr or greater (e.g., at least 5 mL/hr or greater, at least 10 mL/hr or greater, at least 25 mL/hr or greater, at least 50 mL/hr or greater, at least 100 mL/hr or greater, at least 200 mL/hr or greater, at least 500 mL/hr or greater, at least 1 L/hr or greater, at least 5 L/hr or greater, at least 10 L/hr or greater).

In some aspects, a volumetric flow rate of the blood is from 1 mL/hr to 5 L/hr (e.g., from 1 mL/hr to 1 L/hr, from 1 mL/hr to 500 L/hr, from 10 mL/hr to 75 mL/hr, from 20 mL/hr to 50 mL/hr, or from 40 mL/hr to 50 mL/hr).

In some aspects, the volume of blood in the in-line filtration system is 500 mL or less (e.g., 400 mL or less, 300 mL or less, 200 mL or less, 100 mL or less, 50 mL or less, 40 mL or less, 30 mL or less, 20 mL or less, 10 mL or less, or 5 mL or less).

In some aspects, the cell clusters comprise blood clots.

The details of one or more aspects of the disclosure are set forth in the accompanying drawings and the description below. Other features, objects, and advantages of the disclosure will be apparent from the description and drawings and from the claims.

### DESCRIPTION OF DRAWINGS

The skilled person in the art will understand that the drawings described below are for illustration purposes only.

FIGS. 1A-1C show example configurations of the system for the in-line removal of cell clusters from a bodily fluid of a subject according to the present disclosure.

FIG. 2 shows example high-flow filter assemblies using a net mesh design.

FIGS. 3A-3B shows example high-flow filter assemblies using a semi-porous microwell design.

FIGS. 4A-4C show top and side views of example net mesh filter designs having no mesh supports (FIG. 4A), co-planar supports (FIG. 4B), and raised supports (FIG. 4C)

FIGS. 5A-5C show example configurations of bubble removers configured for use in the present systems and methods.

FIG. 6 shows filtering elements in hexagonal and square patterns.

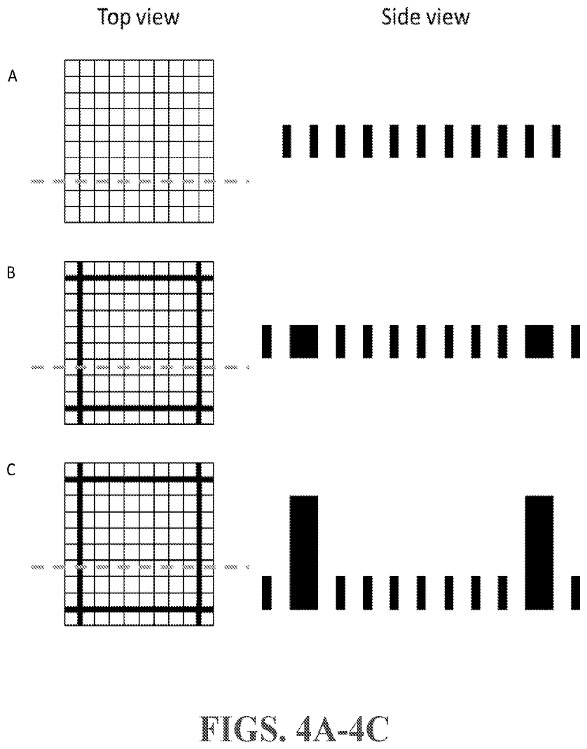

FIGS. 7A-7D shows images of example molds and filter substrates for in-line filtration use.

FIG. 8 shows blood damage from the device measured by checking hemolysis levels before and after the process.

FIG. 9 shows a schematic of an example extracorporeal system developed for in-line, longitudinal enrichment of CTC clusters from rat models.

FIGS. 10A-10C show components of an example extracorporeal system. (FIG. 10A) Assembled system components to be used for characterization studies. (FIG. 10B) Liquid crystal display that shows the operational information. (FIG. 10C) The printed circuit board that controls the individual units of the system.

FIG. 11 shows photographs of the example system. System components are buried under heat conductive beads (not shown) for homogeneous distribution of temperature.

FIG. 12 shows an overview of a filter used for in-vivo, in-line filtration showing the isolated cancer cells. (GFP marker of MDA-MB-231 injected cells, DAPI).

FIG. 13 shows an-vivo study of the device suggests its therapeutic potential. 10 minutes of running the device with both SRG and RNU rats made a huge difference in the cancer tumor load delivered.

FIG. 14 shows the experimental setup used for testing the developed extracorporeal system.

FIG. 15 shows fluorescence microscope images of the control and processed blood cells (RBCs, WBCs). The images show the morphology of the cells. For identification purposes, WBCs were stained using PE-CD45 (TRITC) conjugated antibody. Scale bars, 20 μm.

FIG. 16 shows a viability assessment of blood cells after being processed with the example filter assembly.

FIG. 17 shows a sample fabricated net filter formed as a flat thin film.

FIG. 18 shows a schematic illustration of a fabrication process involving soft lithography and micromolding-based techniques for the realization of polymer devices from reusable molds in a laboratory environment.

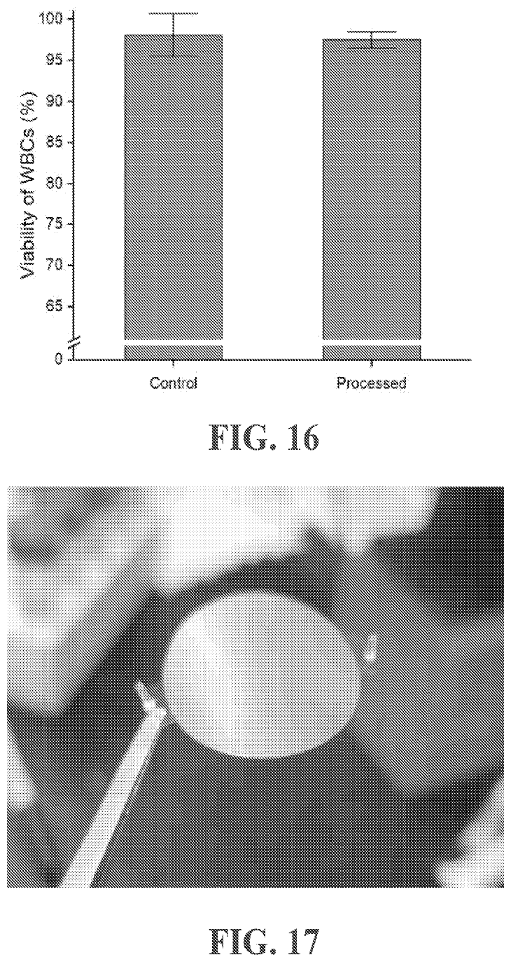

FIG. 19 shows a high-flow filter assembly having an integrally formed sealing region.

FIG. 20 shows examples of stacking the filters. Left: stacking filter holders with different size filters. Right: Stacking filters in a filter holder.

FIG. 21A-21E show (FIG. 21A) compliance with a commercial hemodynamics machine. (FIG. 21B) The panel and plug-and-play design make it easy to use. (FIG. 21C) The device being used for therapeutic purposes. (FIG. 21D) Multiple devices being used for multiple subjects in parallel. (FIG. 21E) Consumables of the system which are sterilized and ready to use.

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce,” or “abrogate,” (used interchangeably) or other forms of the word, such as “reducing” or “reduction,” or “abrogating” or “abrogation” is meant lowering of an event or characteristic. It is understood that this is typically in relation to some standard or expected value, in other words it is relative, but that it is not always necessary for the standard or relative value to be referred to.

By “increase” or other forms of the word, such as “increasing,” is meant raising or elevating. It is understood that this is typically in relation to some standard or expected value, in other words it is relative, but that it is not always necessary for the standard or relative value to be referred to.

As used herein, by a “subject” is meant an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Control” refers to a sample or standard used for comparison with an experimental sample. In some embodiments, the control is a sample obtained from a healthy subject (or a plurality of healthy subjects), such as a subject or subjects not expected or known to have a particular polymorphism. In additional embodiments, the control is a historical control or standard reference value or range of values (such as a previously tested control sample or plurality of such samples), or group of samples that represent baseline or normal values. A positive control can be an established standard that is indicative of a specific methylated nucleotide. In some embodiments a control nucleic acid is one that lacks a particular methylated nucleotide, and is used in assays for comparison with a test nucleic acid, to determine if the test nucleic acid includes the methylated nucleotide.

“Detecting” is used herein to identify the existence, presence, or fact of something. General methods of detecting are known to the skilled artisan and may be supplemented with the protocols and reagents disclosed herein. For example, included herein are methods of detecting a nucleic acid molecule in sample. Detection can include a physical readout, such as fluorescence output.

An “isolated” biological component (such as a nucleic acid molecule) has been substantially separated, produced apart from, or purified away from other biological components. Nucleic acid molecules which have been “isolated” include nucleic acids molecules purified by standard purification methods, as well as those chemically synthesized. Isolated does not require absolute purity, and can include nucleic acid molecules that are at least 50% isolated, such as at least 75%, 80%, 90%, 95%, 98%, 99% or even 100% isolated.

A “sample,” such as a biological sample, is a sample obtained from a subject. As used herein, biological samples include all clinical samples useful for detection of a methylated nucleotide, including, but not limited to, cells, tissues, and bodily fluids, such as: blood; derivatives and fractions of blood, such as serum; urine; sputum; or CVS samples. In a particular example, a sample includes blood obtained from a human subject, such as whole blood or serum. As used herein, the term “whole blood” refers to blood comprising blood plasma, which is typically unclotted, and cellular components. The plasma represents about 50 to about 60% of the volume, and cellular components, i.e. erythrocytes (red blood cells, or RBCs), leucocytes (white blood cells, or WBCs), and thrombocytes (platelets), represent about 40 to about 50% of the volume. Reference to “whole blood” may refer to whole blood of an animal, such as whole blood of a human subject.

The terms “blood plasma” or “plasma” refer to the liquid part of the blood and lymphatic fluid, which makes up about half of the volume of blood (e.g. about 50 to about 60 vol.-%). Plasma is devoid of cells, and unlike serum, has not clotted. So it contains all coagulation factors, in particular fibrinogen. It is a clear yellowish liquid comprising about 90 to about 95 vol.-% water.

As used herein, the term “hemolysis” refers to the rupture of erythrocytes, e.g. due to chemical, thermal or mechanical influences, causing the release of the hemoglobin and other internal components into the surrounding fluid.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer, anal cancer; angiosarcoma (e.g., lymphangiosarcoma, lymphangioendotheliosarcoma, hemangiosarcoma); appendix cancer, benign monoclonal gammopathy; biliary cancer (e.g., cholangiocarcinoma); bladder cancer, breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer, gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer, inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer, myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer, rhabdomyosarcoma; salivary gland cancer, skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer, sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer, vaginal cancer, and vulvar cancer (e.g., Paget's disease of the vulva).

### Methods and Systems

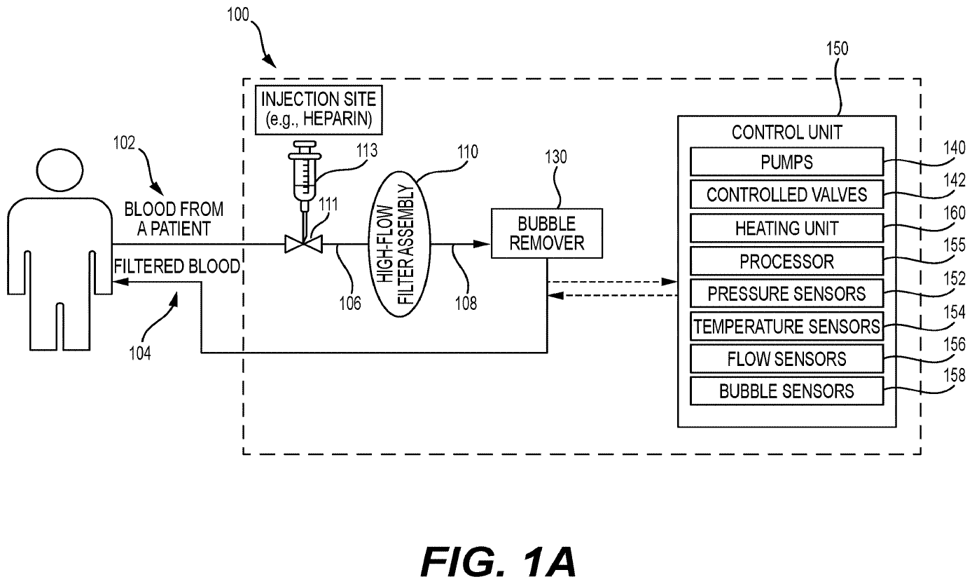

FIG. 1A shows an exemplary system 100 for the in-line removal of cell clusters from a bodily fluid (e.g., blood sample) of a subject. The system 100 receives a bodily fluid 102 from the subject, where it can be moved towards a high-flow filter assembly 110. In situations where the fluid is blood, the fluid can be retrieved from the subject by setting an arterial or venous line, for example, with a carotid artery, femoral artery, jugular vein, common femoral vein and subclavian veins. In adult humans, blood is typically withdrawn and circulated through the system at a flow rate of 50 mL/min to 600 mL/min, however, the present system freely allows for a variety of flow rates. In some examples, a volumetric flow rate of the blood is 1 mL/hr or greater (e.g., at least 5 mL/hr or greater, at least 10 mL/hr or greater, at least 25 mL/hr or greater, at least 50 mL/hr or greater, at least 100 mL/hr or greater, at least 200 mL/hr or greater, at least 500 mL/hr or greater, at least 1 L/hr or greater, at least 5 L/hr or greater, at least 10 L/hr or greater). In some aspects, a volumetric flow rate of the blood is from 1 mL/hr to 5 L/hr (e.g., from 1 mL/hr to 1 L/hr, from 1 mL/hr to 500 L/hr, from 10 mLUhr to 75 mL/hr, from 20 mL/hr to 50 mL/hr, or from 40 mL/hr to 50 mLUhr). In some aspects, the volume of blood in the in-line filtration system is 500 mL or less (e.g., 400 mL or less, 300 mL or less, 200 mL or less, 100 mL or less, 50 mL or less, 40 mL or less, 30 mL or less, 20 mL or less, 10 mL or less, or 5 mL or less).

The fluid 102 retrieved from the subject is circulated to a high-flow filter assembly 110 where cell clusters (e.g., circulating tumor cell (CTC) clusters) can be removed. The system and high-flow filter assemblies provided herein advantageously show high-throughput filtering of cell clusters while inducing low fluidic stress (e.g., to reduce hemolysis levels).

The system 100 further includes an injection site 111 where an agent 113 (e.g., an anticoagulant) can be mixed with the circulating blood before or after removal from the subject. In various aspects, the agent is one or more anticoagulants. Non-limiting examples of suitable anticoagulants include heparin, ethylenediaminetetraacetic acid (EDTA), EDTA disodium salt, EDTA tetrasodium salt, EDTA dipotassium salt, EDTA diammonium salt, ethylenebis(oxyethylenenitrilo)tetraacetic acid (EGTA), EDTA trisodium salt, EDTA tripotassium salt, ethylene glycol-O,O-bis(2-aminoethyl)-N,N,N,N-tetraacetic acid, N-(2-hydroxyethyl)ethylenediamine-N,N,N-triacetic acid trisodium salt, citrate, acid-citrate-dextrose, di-ammonium hydrogen citrate, di-ammonium tartrate, warfarin, N-(2-bis(carboxymethyl)aminoethyl)-N-(2-hydroxyethyl)glycin salt dihydrate, citric acid, citric acid monosodium salt, citric acid disodium salt, citric acid trisodium salt, citric acid monopotassium salt, citric acid tripotassium salt, protein C/protein S, nitrilotriacetic acid, potassium sodium tartrate, potassium hydrogen D-tartrate, L-tartaric acid monosodium salt, L-tartaric acid disodium salt, L-tartaric acid dipotassium salt, streptokinase, protamine sulfate, tris(carboxymethyl)amine, anti-thrombin III, phenprocoumon, hirudin, nicoumalone, Coumadin, glycosaminoglymays, ibuprofen, acetylsalicylic acid, indomethacin, prostaglandins, sulfinpyrazone, urokinase, hirulog, tissue plasminogen activator, coumarin, or combinations thereof.

In some examples, the agent can include a detectable label for facilitating diagnostics. The detectable label can include, for example, radioactive labels, luminescent labels, fluorescent dyes, compounds having an enzymatic activity, magnetic labels, antigens, and compounds having a high binding affinity for a detectable label.

Although the injection site 111 in the system 100 is shown before the high-flow filter assembly 110, it can be advantageous to position an injection site after the fluid has been filtered. For example, an injection site can be positioned in order to deliver a therapeutic agent to the filtered fluidic product. Non-limiting examples of therapeutic agents include immuno-suppressants, anti-inflammatories, anti-proliferatives, anti-migratory agents, anti-fibrotic agents, proapoptotics, vasodilators, calcium channel blockers, anti-neoplastics, anti-cancer agents, antibodies, anti-thrombotic agents, anti-platelet agents, IIb/IIa agents, antiviral agents, mTOR (mammalian target of rapamycin) inhibitors, non-immunosuppressant agents, and combinations thereof.

In some examples, the agent includes an anti-cancer agent. abraxane; ace-11; acivicin; aclarubicin; acodazole hydrochloride; acronine; adozelesin; aldesleukin; altretamine; ambomycin; ametantrone acetate; amrubicin; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; herceptin; hydroxyurea; idarubicin hydrochloride; ifosfamide; ilmofosine; iproplatin; irinotecan; irinotecan hydrochloride; lanreotide acetate; lapatinib; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; romidepsin; safingol; safingol hydrochloride; semustine; simtrazene; sparfosate sodium; sparsomycin; spirogermanium hydrochloride; spiromustine; spiroplatin; stem cell treatments such as PDA-001; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs may be used, including, but not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; b-FGF inhibitor, bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor, carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor, mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (GENASENSE®); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor, platinum complex; platinum compounds; platinum-triamine complex; porfimer sodium; porfiromycin; prednisone; propyl bis-acridone; prostaglandin J2; proteasome inhibitors; protein A-based immune modulator, protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor, retelliptine demethylated; rhenium Re 186 etidronate; rhizoxin; ribozymes; RH retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur, tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

The system 100 further includes a control unit 150 which can monitor and affect system parameters such as flow rate of the fluid, system temperature, agent delivery rate, and flow direction. In various examples, the control unit 150 can contain various pumps, pressure monitoring devices, valves, electronic components, connector fittings, tubing, etc., to coordinate the operation of the other system components. The control unit 150 uses sensor data to operate the system 100 in a feedback-controlled manner which can allow for constant monitoring of the data from the sensors and real-time adjustments to the device's operation accordingly. In some aspects, the system uses a look-up table control model where the control data is pre-fitted and the controller is set to run on a flow rate. In this case the flow sensor is used to independently monitor the flow. These controls ensure that the system 100 is always operating within its safe limits. The control unit 150 can also control the smooth ramp-up and ramp-down of the flow to ensure a shock-free start and end to the operation. This is significant for devices that generate a lot of power, as it can prevent damage to the device or the user.

The control unit 150 shown in FIG. 1A includes multiple sensors (i.e., pressure sensors 152, temperature sensors 154, flow sensors 156, bubble sensors 158) that monitor system parameters for control by a processor 155 in the control unit 150. The control unit 150 further includes system controls to effectuate control of the fluid flow through the system 100. For example, a pump 140 is used to move the blood through the system 100. The pump 140 shown in FIG. 1A is a dialysis pump configured to provide a pressure (e.g., positive and/or negative) to circulate the subject's blood through the high-flow filter assembly 110. Various types of pumps can be utilized in the system, for example, rotary lobe pumps, progressing cavity pumps, rotary gear pumps, piston pumps, diaphragm pumps, screw pumps, gear pumps, hydraulic pumps, vane pumps, regenerative (peripheral) pumps, peristaltic pumps, or any combination thereof. In various aspects, a plurality of pumps are used to circulate the fluid through the system.

The processor 155 of the described system 100, can be used for processing information associated with the process(es) or method(s) described herein, may include, for example, any computer processor is known in the art capable of performing calculations and directing functions for interpreting and/or performing input, output, calculation, and display of data in accordance with the disclosed methods. The processor may comprise any type of processing unit, such as typical computer processor(s), controller(s), microcontroller(s), microprocessor s), and/or programmable logic controllers (PLCs). The information to be processed by the processor may include, for example, information contained in analog or digital signals and/or translated signals and/or information contained in a data storage. Processing of the information may involve, for example, performing calculations on received signals such as, but not limited to, vector analysis, picture identification, pattern recognition, frequency analysis/Fourier transforms, numerical computations, machine learning, or, as described herein, applying predetermined or recursively fit correlative algorithms to received sensor data. In some embodiments, the system comprises more than one processor, and the reference herein to “processor” includes reference to multiple processors and vice versa.

The system 100 may also include a display 142 (which may be co-located with the processor 155 in the control unit 150, e.g., where the processor and display are part of a computer or server used for carrying out the method steps described herein) for visually presenting information associated with the described methods. The display may comprise, for example, a computer monitor (e.g., LCD, a CRT monitor, a projection (e.g., heads-up display (HUD) laser), etc. In some embodiments, the visual display may comprise, for example, that of a mobile device such as a tablet computer, cellular phone, smartphone, personal digital assistant (PDA), personal computer (PC), laptop computer, augmented reality display (e.g., Google™ Glass™ or Microsof™ HoloLens™), etc. The information presented on the display may include measurements obtained by or derived from the sensor data, for example, and/or may include any other information collected in the course of carrying out the methods described herein, prompts for information entry associated with one or more steps of the described methods, and/or any predetermined formulae or algorithms, as previously described. The display may also be capable of receiving input (such as, e.g., where the display includes a touch-screen and is capable of receiving touch input and accordingly transmitting information to the processor).

The system can further include an imaging unit configured to image and/or screen retained cell clusters. For example, the imaging unit can include a microscope (e.g., SEM microscope or a fluorescent microscope).

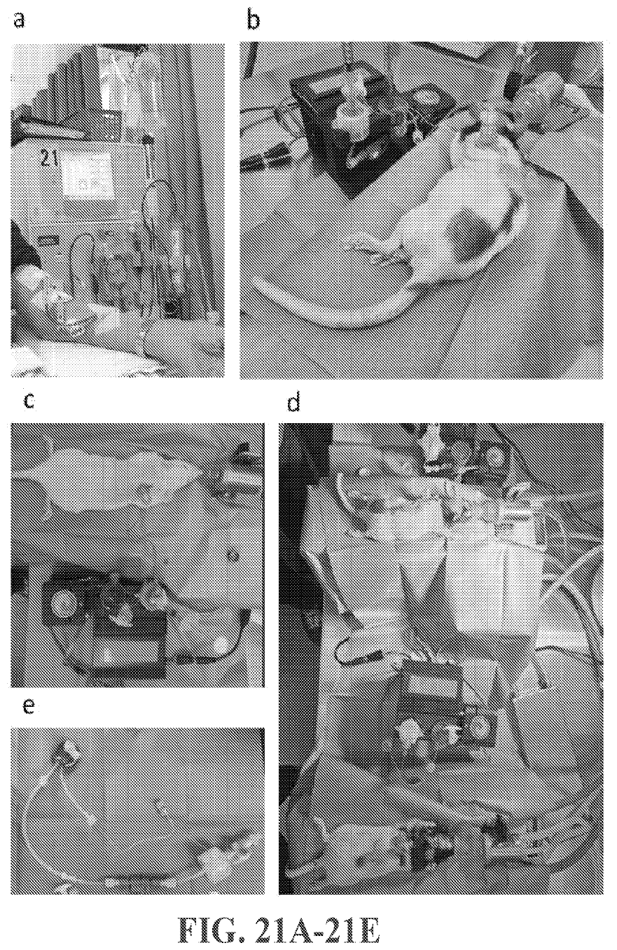

In some aspects, the system is configurable with a panel design that is compatible with commercial hemodialysis machines, such as that shown in FIGS. 21A-21E. In some aspects, the components contacting a fluid (e.g., blood) comprise a sterilizable material. In some aspects, the system comprises disposable components.

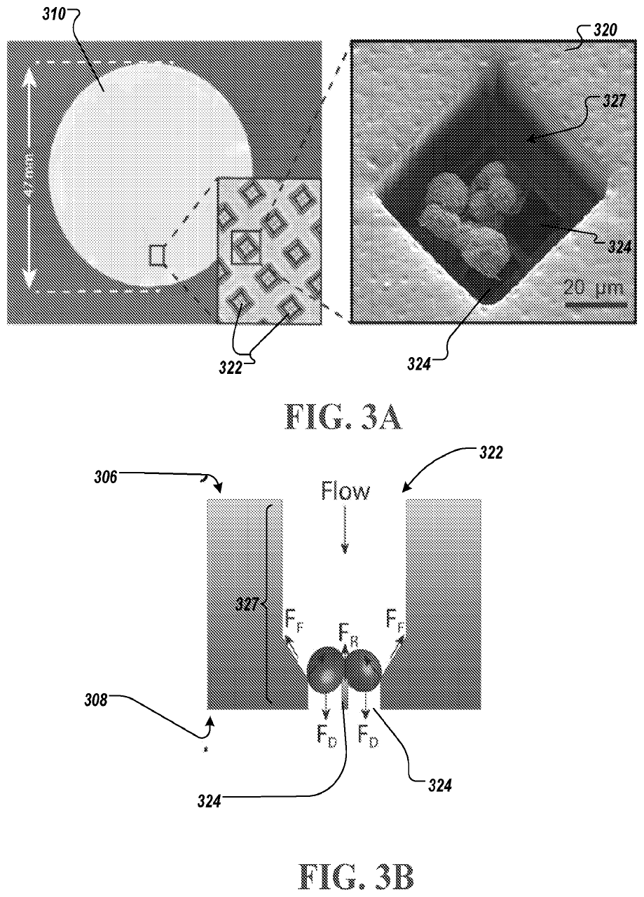

Gases present in blood returned to the subject may enter the blood and cause embolism. Thus, the system 100 further includes a bubble remover 130, such as those shown in FIGS. 5A-5C, to reduce the likelihood of adverse events caused by gases, the bubble remover 130 is configured to remove gases (e.g., air bubbles) from the filtered output. FIG. 5A shows two example configurations of bubble remover suitable for use in the presently described systems. In the example arrangement, the bubble remover 530 includes an inlet stream 532 of the filtered output entering a housing 540 of the bubble remover 530, wherein the inlet stream 532 is configured to contact a substantially immiscible liquid 552 (e.g., saline) having a lower density than the filtered output. The bubble remover 530 in the left side FIG. 5A, the inlet stream 532 is configured to enter the bubble remover 530 substantially towards a side wall 542 of the housing 540. The bubble remover can also serve as a damper, reducing the pulsative nature of the fluid caused by the pump, resulting in a more uniform flow going back to the subject. In some aspects, as shown in the left side of FIG. 5A, the inlet stream 532 is configured to enter the bubble remover 530 substantially away from a side wall 542 of the housing 540.

The system 100 further includes a heating unit 160 which is configured to provide thermal energy to the fluid inlet 102 and/or the filtered output 104. In some aspects, the heating unit is configured to supply thermal energy to at least some of the component of the system. For example, heat can be supplied to components, such as tubing used to contain the fluid as it is circulated, such that fluid inlet 102 and/or the filtered output 104 are heated to a desired temperature (e.g., body temperature). The heating unit 160 is controlled by the control unit 150 based on signals recorded by the temperature sensors 154. Temperature control using the heating unit may be realized using any heating and cooling mechanism known in the art. In some examples, the heating unit comprises a thermal lamp and/or a blood warmer.

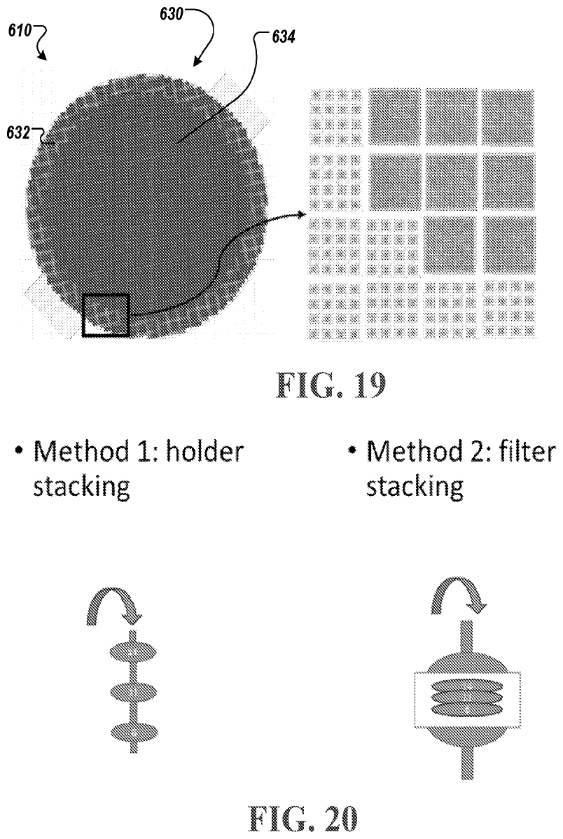

Although the system 100 in FIG. 1A shows a single high-flow filter assembly 110, other configurations may also be used. For example, the system 100b in FIG. 1B includes a plurality of high-flow filter assemblies (110a, 110b) positioned in series. Filter assemblies having different size openings can be arranged in series to separate cell clusters by size or to provide further removal of particulates. Multiple filters having different sizes can be arranged in separate filter holders, or stacked in a single filter holder as shown in FIG. 20. In yet another arrangement, as shown in FIG. 1C, a plurality of filter-assemblies (110a, 110b) are positioned in parallel. When arranged in parallel fluidic circuits, filter assemblies can be replaced by changing the fluid path using valves controlled by the control unit without disrupting the operation. In some examples, parallel fluidic circuits can be used to switch between filters (e.g., filters having different sizes). In some aspects, the system includes high-flow filter assembly having a net-mesh design. In some aspects, the system includes a high-flow filter assembly having a semi-porous microwell design. A summary of several tested properties for filter assemblies using the designs provided in Examples 1-2 is shown below in Table 1. The properties of the various filter assemblies included in Table 1 are illustrative and are not intended to limit the scope of the present disclosure.

TABLE 1
Filter properties of example arrangements on a 25 mm diameter filter holder.

| | | | | | | | Nominal | | | |
|---|---|---|---|---|---|---|---|---|---|---|
| | | | | | | | flowrate | | | |
| | | | | | | Relative | based on | | | |
| | | | | | Surface | SUR - | Microwells | | | |
| | Linear | opening | | Total | utilization | Normal | technology | | 10 mL | |
| | size | size | Period | wells | rate | to full | nominal | | process | |
| Name | (#) | (um) | (um) | (*openings) | (SUR) | net | rate. | | time | |
| Microwell | 227 | 13.5 | 110 | 40.45k (162k*) | 6.02% | 7.94% | 6.25 | mL/hr | 96 | min |
| 10*10 net | 142 | 13.5 | 181 | 1.58M* | 55.63% | 73.33% | 60.97 | mL/hr | 9.8 | min |
| 20*20 net | 76 | 13.5 | 336 | 1.81M* | 64.57% | 85.12% | 69.53 | mL/hr | 8.6 | min |
| 25*25 net | 62 | 13.5 | 413.5 | 1.86M* | 66.62% | 87.82% | 71.23 | mL/hr | 8.4 | min |
| 30*30 net | 52 | 13.5 | 491 | 1.91M* | 68.04% | 89.69% | 72.43 | mL/hr | 8.3 | min |
| Full net | 1612 | 13.5 | 15.5 | 2.04M* | 75.86% | 100.00% | 78.72 | mL/hr | 7.6 | min |

| | | | | | | | |
|---|---|---|---|---|---|---|---|
| Ring net | 13.5 | 1.12M* | 41.53% | 54.7% | 43.125 | 13.91 | min |

Also disclosed herein are methods for the in-line removal of cell clusters. In some aspects, the method includes: causing a volume of a fluid of a subject (e.g., blood) to circulate within a system, wherein the system includes: a high-flow filter assembly having a filter substrate that forms a plurality of filtering elements (e.g., semipermeable microwells), the filter assembly being configured to receive (e.g., directly) an inlet fluid (e.g., unfiltered blood) from a subject at a first side and output a filtered output at a second side, wherein each filtering element defines a plurality of apertures extending between the first side and the second side of the filter, each filtering element forming a cluster-exclusion barrier in a trapping region of the filtering element to substantially prevent cell clusters of multiple cells (e.g., circulating tumor cell (CTC) clusters) from passing from the first side to the second side. In some aspects, the cell clusters are blood clots.

In some aspects, the method further includes causing the filtered output to be returned to the subject. The filtered output can be returned to the subject using, for example, a blood pump to direct the filtered output to a veinous line of subject. In some aspects, the method also includes isolating retained cell clusters from the high-flow filter assembly.

In some aspects, the cell clusters are retrieved by flowing a recovery fluid (e.g., PBS) through the high-flow filter assembly in a flow direction from the second side to the first side. By flowing the recovery fluid in the reverse direction, cell clusters can become dislodged from the high-flow filter assembly where they may be collected in a holding container for further analysis.

In some aspects, the isolated cell clusters are used to diagnose, quantify, characterize, and/or monitor a cancer in the subject. In some aspects, the cancer is selected from the group consisting of prostate cancer, ovarian cancer, breast cancer, and medulloblastoma. For example, isolated clustered particles can be imaged (e.g., using an imaging element) and subjected to any form of molecular and function analysis. By analyzing the clustered particles, valuable information about the clustered particles can be obtained, including the origin of cancer and mutations of cells. In some aspects, the method further includes starting, suggesting, adjusting, and/or directing a treatment parameter in response to the isolated cell clusters.

In some aspects, a volumetric flow rate of the fluid through the high-flow filter assembly is 1 mL/hr or greater (e.g., at least 5 mL/hr or greater, at least 10 mL/hr or greater, at least 25 mL/hr or greater, at least 50 mL/hr or greater, at least 100 mL/hr or greater, at least 200 mL/hr or greater, at least 500 mL/hr or greater, at least 1 L/hr or greater, at least 5 L/hr or greater, at least 10 L/hr or greater). In some aspects, a volumetric flow rate of the fluid is from 1 mL/hr to 100 mL/hr (e.g., from 10 mL/hr to 75 mL/hr, from 20 mL/hr to 50 mL/hr, or from 40 mL/hr to 50 mL/hr). In some aspects, the volume of blood in the in-line filtration system is 500 mL or less (e.g., 400 mL or less, 300 mL or less, 200 mL or less, 100 mL or less, 50 mL or less, 40 mL or less, 30 mL or less, 20 mL or less, 10 mL or less, or 5 mL or less). Advantageously, the present high-flow filter assemblies allow for the use of low dead volumes, ensuring that the systems and methods are transferrable to both large and small animals.

Also disclosed herein are kits including the components of the systems described herein. In some aspects, the kit include a high-flow filter assembly having a micro-structure filter substrate that forms a plurality of filtering elements (e.g., semipermeable microwells). The filter assembly is configured to receive (e.g., directly) an inlet fluid (e.g., unfiltered blood) from a subject at a first side and output a filtered output at a second side, wherein each filtering element defines a plurality of apertures extending between the first side and the second side of the filter, each filtering element forming a cluster-exclusion barrier (e.g., a size-exclusion barrier) in a trapping region of the filtering element to substantially prevent cell clusters of multiple cells (e.g., circulating tumor cell (CTC) clusters) from passing from the first side to the second side. In some aspects, the kit further includes tubing. In some aspects, the kit further includes holding containers (e.g., test tubes) for containing isolated cell clusters and/or fluid. In some aspects, one or more of the components of the kit are disposable. In some examples, one or more of the components of the kit are sterilizable. In some aspects, the components of the kit are readily adaptable to be used on commercial hemodialysis machines.

### Example 1: Example System for the in-Line Removal of Cell Clusters Using Net Filter

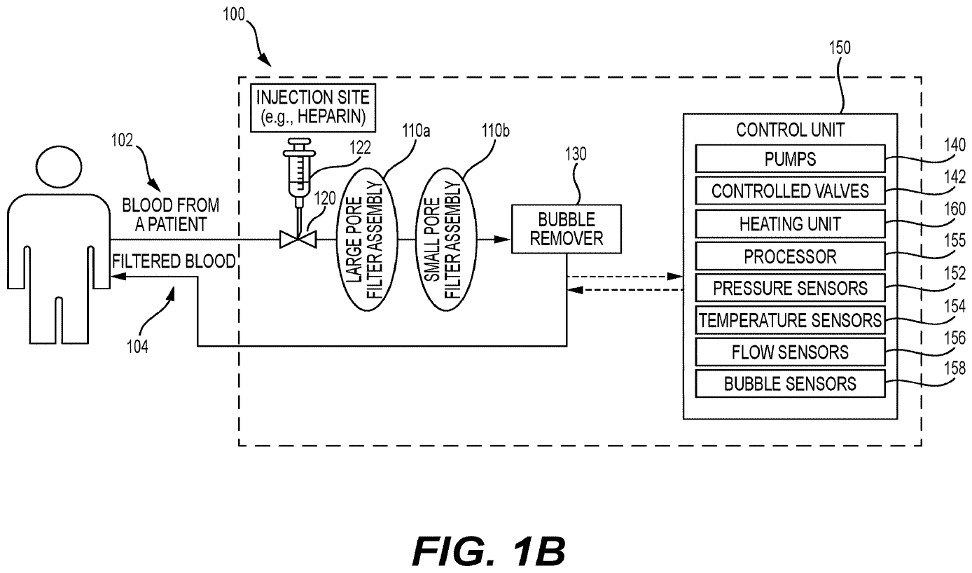

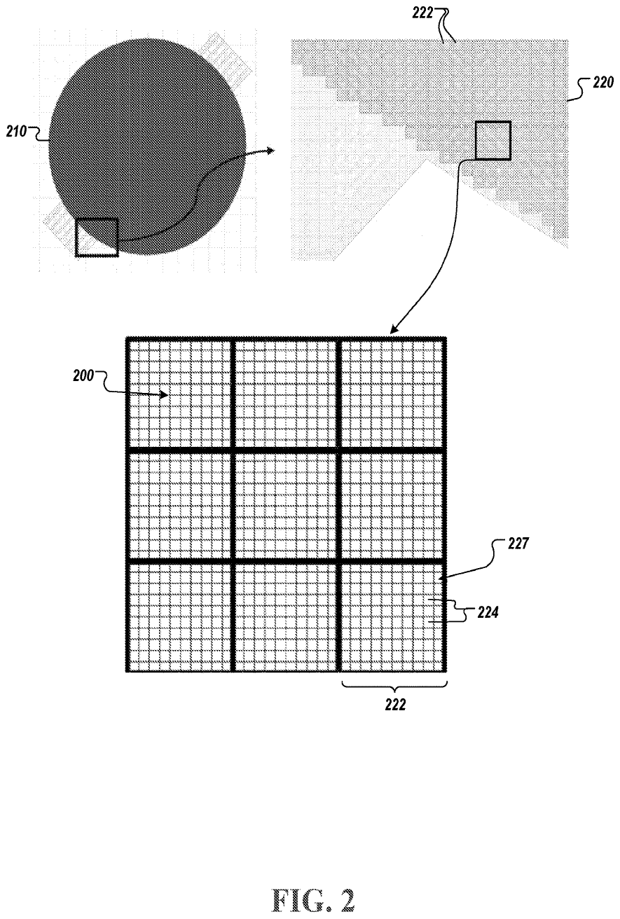

FIG. 2 shows an example of a high-flow filter assembly used in a system according to the present disclosure. The high-flow filter assembly 210 includes a filter substrate 220 that forms a plurality of filtering elements 222. The high-flow filter assembly 210 is configured to receive (e.g., directly) an inlet fluid (e.g., unfiltered blood) from a subject at a first side and output a filtered output 104 at a second side 108, wherein each filtering element 222 defines a plurality of apertures 224 extending between the first side and the second side of the high-flow filter assembly 210. Each filtering element 222 forms a cluster-exclusion barrier 225 in a trapping region 227 of the filtering element 222 to substantially prevent cell clusters of multiple cells from passing from the first side to the second side. The high-flow filter assembly 210 of FIG. 2 depicts a plurality of filtering elements 222 in the form of a supported mesh structure, such as that shown in FIG. 4B and FIG. 4C. Thus, each filtering element forms a discrete structure separated by a reinforced support. However, other examples, such as those depicted in FIG. 4A, include a full net mesh without any supporting structures. In some aspects, a thickness from of the size measured from the first side to the second side is from 1 μm to 1000 μm (e.g., from 1 μm to 500 μm, from 1 μm to 400 μm, from 1 μm to 300 μm, from 1 μm to 200 μm, from 1 μm to 100 μm, from 1 μm to 50 μm, from 1 μm to 40 μm, from 1 μm to 30 μm, from 1 μm to 20 μm, from 1 μm to 10 μm, or from 5 μm to 10 μm). In some aspects, a width of the support is from 1 μm to 1000 μm (e.g., from 1 μm to 500 μm, from 1 μm to 400 μm, from 1 μm to 300 μm, from 1 μm to 200 μm, from 1 μm to 100 μm, from 1 μm to 50 μm, from 1 μm to 40 μm, from 1 μm to 30 μm, from 1 μm to 20 μm, from 1 μm to 10 μm, or from 5 μm to 10 μm). In some aspects, the high-flow filter assembly has a three-dimensional shape (e.g., a conical structure).

The term “net-mesh” refers to an interconnected arrangement of a filter substrate that form a plurality of apertures. In some examples, the net-mesh structure forms a grid array having evenly spaced apertures within a filtering element such as that shown in FIG. 2. Reference to a “full net” means that the plurality of filtering elements are arranged to form a continuous array of apertures (i.e., without spaces between adjacent filtering elements). In some examples, a filter assembly is referred to as an X*Y net design, which defines the number of apertures in a single rectangular filtering element. For example, a 10*10 net includes 10 apertures along an x-direction and 10 apertures along a y-direction. In some examples, X and Y are independently integers greater than 2. In some examples, X and Y are independently integers between 2 and 1000 (e.g., from 2 to 500, from 2 to 400, from 2 to 300, from 2 to 200, from 2 to 100, from 2 to 50, from 2 to 40, from 2 to 30, from 2 to 20, from 2 to 10, from 2 to 100, from 5 to 100, from 10 to 100, from 20 to 100, from 10 to 20, or from 10 to 30). In some examples, the filter assembly comprises a 10*10 net pattern. In some examples, the filter assembly comprises a 20*20 net pattern. In some examples, the filter assembly comprises a 25*25 net pattern. In some examples, the filter assembly includes a 30*30 net pattern. In some examples, the filter assembly comprises a full net pattern.

In some aspects, a surface utilization rate of the filter substrate is 5% or more (e.g., 10% or more, 15% or more, 20% or more, 25% or more, 30% or more, 35% or more, 40% or more, 45% or more, or 50% or more). The surface utilization rate is a measure of the amount of surface area of a filter substrate covered by filtering elements. In some aspects, a surface density of the apertures on the filter substrate is 500 apertures/mm² or more (1000 apertures/mm² or more, 1500 apertures/mm² or more, 2000 apertures/mm² or more, 2500 apertures/mm² or more, or 3000 apertures/mm² or more). In some aspects, each of the filtering elements has a size of from 1 μm to 1000 μm (e.g., from 1 μm to 900 μm, from 1 μm to 800 μm, from 1 μm to 700 μm, from 1 μm to 600 μm, from 1 μm to 500 μm, from 1 μm to 400 μm, from 1 μm to 300 μm, from 1 μm to 200 μm, from 1 μm to 100 μm, from 1 μm to 50 μm, from 5 μm to 40 μm, from 5 μm to 30 μm, or from 5 μm to 20 μm).

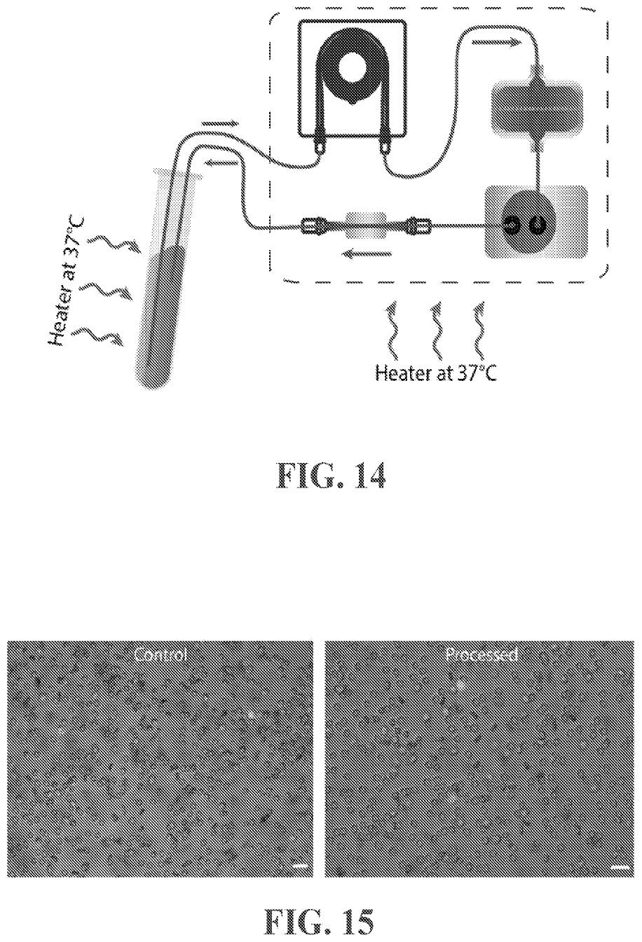

Filtering elements can therefore be formed into a continuous section of apertures separated by a cluster-exclusion barrier. Although each of the filtering elements in the mesh structures of FIGS. 4A-4C has a square shape, other shapes can also be used. For example, the filter elements and/or plurality of apertures can have a triangular, circular, or hexagonal shape. In some examples, the high-flow filter assembly is substantially flat, such as the one shown in FIG. 17. The term “substantially flat” refers to filters having a major surface with a planar 3-dimensional shape. For example, the surface can still be substantially flat if undulations or indentations on the surface are in the range of 1 μm to 1000 μm. The term also encompasses structures comprising more than one flat area arranged in an angle or a curved, e.g., convex or concave configuration, laying flat on the filter and preferably relates to material that is adapted to lay flat on or against the second side of the filter. In some examples, the high-flow filter assembly has a thickness of 1 μm to 1000 μm (e.g., from 1 μm to 500 μm, from 1 μm to 400 μm, from 1 μm to 300 μm, from 1 μm to 200 μm, from 1 μm to 100 μm, from 1 μm to 50 μm, from 1 μm to 40 μm, from 1 μm to 30 μm, from 1 μm to 20 μm, from 1 μm to 10 μm, or from 5 μm to 10 μm). In some aspects, the high-flow filter assembly has a three-dimensional shape (e.g., a conical structure). In other examples, the high-flow filter assembly has a three-dimensional shape (e.g., a conical structure). For example, the high-flow filter can be arranged as a funnel to direct fluid flow to specific regions of the filter.

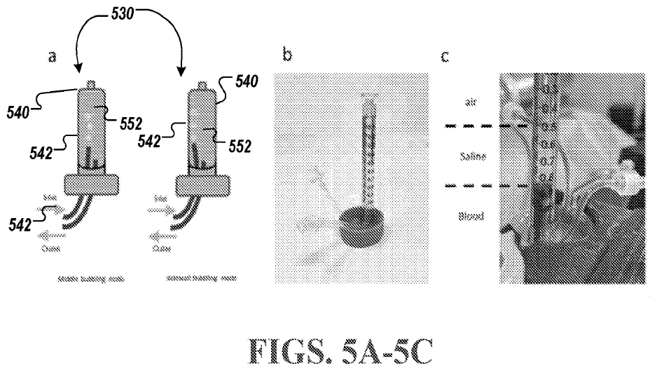

FIGS. 7A-7D each show SEM images of example net-mesh filters and molds used to produce the net-mesh filter according to the present disclosure. The elements in the system can further use a combination of sterilizable and disposable components. In this regard, the in-line detection system can be reused at low-cost with a decreased risk for the development of infections after use. For example, the high-flow filter assembly and other components having direct contact with the fluid sample can include materials that are able to be sterilized using methods such as Ethylene Oxide Sterilization, autoclaving and UV radiation.

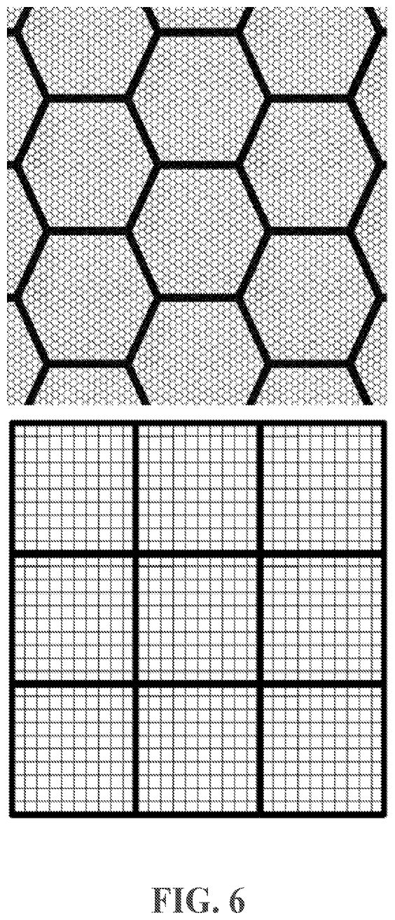

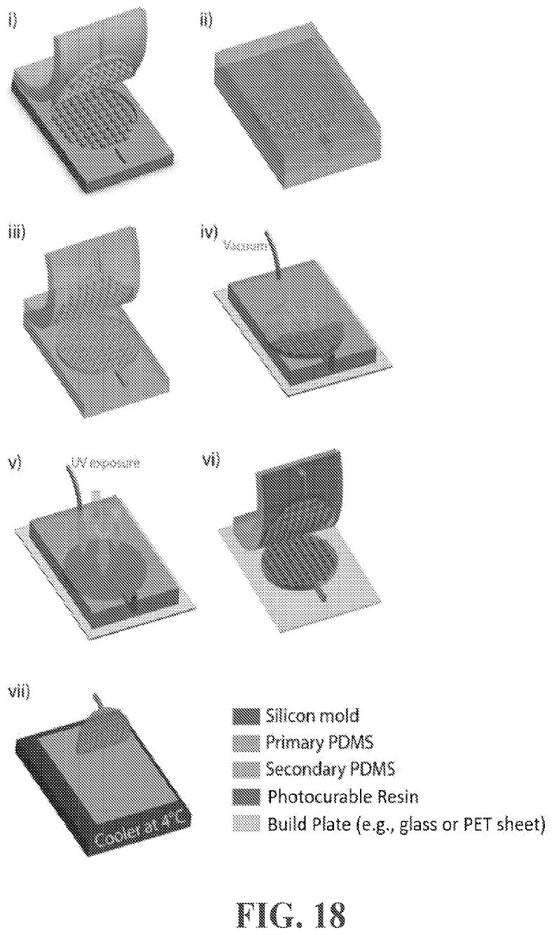

In some aspects, the filter substrate of the high-flow filter assembly is coupled to a filter holder. The term “filter holder” broadly encompasses any receptacle configured to receive and affix the filter substrate in place during operation. Referring now to FIG. 19, the high-flow filter assembly 610 includes a filter holder 630 comprising a sealing region 632, wherein the sealing region 632 defines an area 634 (e.g., a circle) enclosing at least a portion of the high-flow filter assembly 610 with the plurality of filtering elements 622. The sealing region 632 is configured to substantially prevent the inlet fluid from passing through the filter substrate outside of the area and is substantially non-porous to the inlet fluid. The inner part of the filter shown in the area 634 is designed in a way that would capture most cells while the outer part shown as the sealing region 632 is designed to fully seal with an O-ring to accommodate higher pressure buildups. The filter holder 630 of FIG. 19 is integrally formed on the filter substrate. An SEM image of a mold and filter using a ring design is shown in FIG. 7B. In some aspects, the filter holder is a separate element from the filter substrate. The filter holder may also include a combination of integrally formed and separate elements. FIG. 8 shows minimal blood damage from the device measured by checking hemolysis levels before and after the process.

To achieve the desired feature resolution of the net-mesh design high-flow filter assembly, a negative mold was first fabricated similar to that used in Example 2. Here, the mold was formed using a 3-mask microfabrication process that involved thin film deposition on a build plate (e.g. glass build plate). In the disclosed example, Surgical Guide Resin, a 3D printable resin from Formlabs, was chosen as the build resin due to its ability to fabricate intricate features, its biocompatibility, sterilizability, structural resilience, and non-autofluorescence. Despite being a 3D printable resin configured for resolution on the scale of feature sizes ~50 μm, the present study surprisingly found that the material was able to sufficiently produce structures having a feature size between 1-3 μm. The material also allowed for added control of the resin's viscosity via temperature modulation, which enhanced microchannel filling. The resin was then added to the resulting mold and heated on the glass build plate to a temperature of 45-55° C. to decrease viscosity and enhance flowability of the resin to fill the narrow voids. Subsequently, the resin was cooled to a temperature of 5-15° C. before and during curing with a UV light. Once cured with UV light, the resulting filter was washed with isopropyl alcohol to remove excess resin and heated in a 70° C. oven for 30 formed a high-flow filter structure of the high-flow filter assembly configured for in-line filtration.

### Example 2: Example System for the in-Line Removal of Cell Clusters in Mice Using Microwell Filter

In this study, an extracorporeal circulation system is shown which can be used to overcome the withdrawable blood volume limitation. While depending on the age and weight of the subject, the total blood volume that can be drawn in an 8-week period is limited to 550 mL for heathy human adults weighing more than 110 pounds [1]. Similarly, the sampling volume over a 4-week period is limited to 10% of the total circulating blood volume in healthy mice and rats, which corresponds to ~250 μL and ~2.5 mL for mice and rats, respectively [2]. In this study, an extracorporeal circulation system was designed by considering the rat as the animal model; however, it can be designed for other living-beings.

By using an extracorporeal circulation system, the blood from an artery was sampled, and CTC clusters were isolated from the blood using the Cluster-Wells technology [3] and returned the processed blood back to the rat through a vein, similar to a hemodialysis system used in patients with chronic kidney disease. As the healthy blood cells are not depleted during operation, continuous and longitudinal screening of blood becomes feasible. The possibility of isolating large number of CTC clusters would allow researchers to better study tumor heterogeneity, biological/molecular characterization and ultimately pave the way for more efficient downstream functional analysis such as in vitro culturing and drug testing.

The selection of the artery and vein plays an important role for efficient isolation of CTC clusters. First, they need to be compatible with catheterization for easy and continuous access to the bloodstream of animals. While any vein/artery is an option, most commonly used access sites for the placement of catheters are the carotid artery, femoral artery, jugular vein, common femoral vein and subclavian veins [4], [5]. Among these arteries and veins, carotid artery and jugular vein are the most suitable ones due to their higher volumetric blood flow rate compared to other arteries and veins with ~4-6 mL/min volumetric blood flow rate in rats [6]-[9], where the higher flow rate would facilitate encountering more CTC clusters.

Considering the cardiac output of rats (~48 mL/min) [10], approximately 10% of the circulating blood passes through the carotid artery. Furthermore, one-pass circulation time of blood in mice was previously reported as 15 seconds [11], while half-life of CTC clusters in mouse circulatory system was measured as ~8 minutes [12], meaning that before they are entrapped in capillaries of distal organs, CTC clusters can circulate in average 32 times within the body. By assuming similar circulatory system characteristics in rat models and homogeneous distribution of CTC clusters in circulating blood, we expect to encounter >90% of the CTC clusters at the catheterized carotid artery. Being able to isolate a large portion of CTC clusters in circulation would allow effective utilization of these metastatic precursors in both research and clinical settings.

The designed system is conceptually similar to a hemodialysis system used in patients with kidney disease. In the system, instead of removing the excess water and waste through a specialized membrane, the developed technology Cluster-Wells will be used for removing CTC clusters from the bloodstream of tumor-bearing rats for a longitudinal period of time.

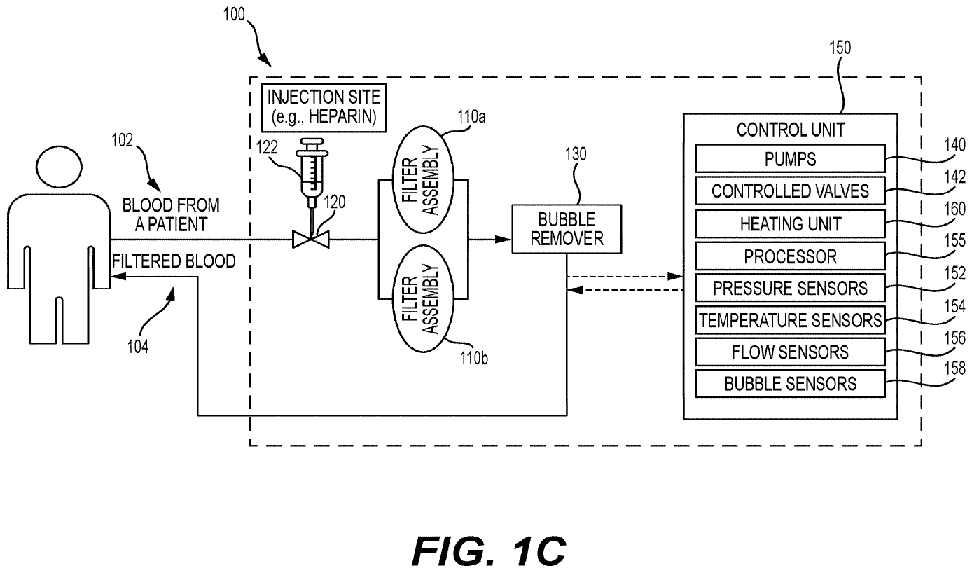

FIGS. 3A-3B shows an example of a high-flow filter assembly 310 having a semi-porous microwell structure. The high-flow filter assembly 310 includes a filter substrate 320 that forms a plurality of filtering elements 322. The high-flow filter assembly 310 is configured to receive (e.g., directly) an inlet fluid (e.g., unfiltered blood) from a subject at a first side 306 and output a filtered output at a second side 308. Each filtering element 322 defines a plurality of apertures 324 extending between the first side and the second side of the high-flow filter assembly 310. Each filtering element 322 forms a size-exclusion barrier 325 in a trapping region 327 of the filtering element 322 to substantially prevent cell clusters of multiple cells from passing from the first side 306 to the second side 308.

The high-flow filter assembly of the present example was formed using a combination of different microfabrication techniques. First, a negative mold of the filter assembly was fabricated involving out of silicon using a 3-mask microfabrication process that involved thin film deposition with wet and reactive ion etching steps. Next, the silicon mold was then transferred into polydimethylsiloxane (PDMS) twice to make the same structure as the silicon mold. A photocurable polymer was then added to the resulting PDMS mold and cured to form a structure of the high-flow filter assembly configured for in-line filtration. An example method of fabricating a mold for a microwell-based filter assembly is shown in FIG. 21.

In some aspects, the filter substrate comprises a biocompatible material. The term “biocompatible material” refers to any material that is biologically compatible by not producing a toxic, injurious, or immunological response in living tissue. For example, the biocompatible material can be a biocompatible polymer, such as polyethylene, low-density polyethylene, polymethylmethacrylate (PMMA), polyetheretherketone (PEEK), polyetherketoneketone (PEKK), and polyurethane. In some aspects, the filter substrate comprises a UV-curable polymer. In some aspects, the filter substrate comprises a printable material (e.g., an acrylate polymer). In some aspects, the filter substrate comprises a disposable material.

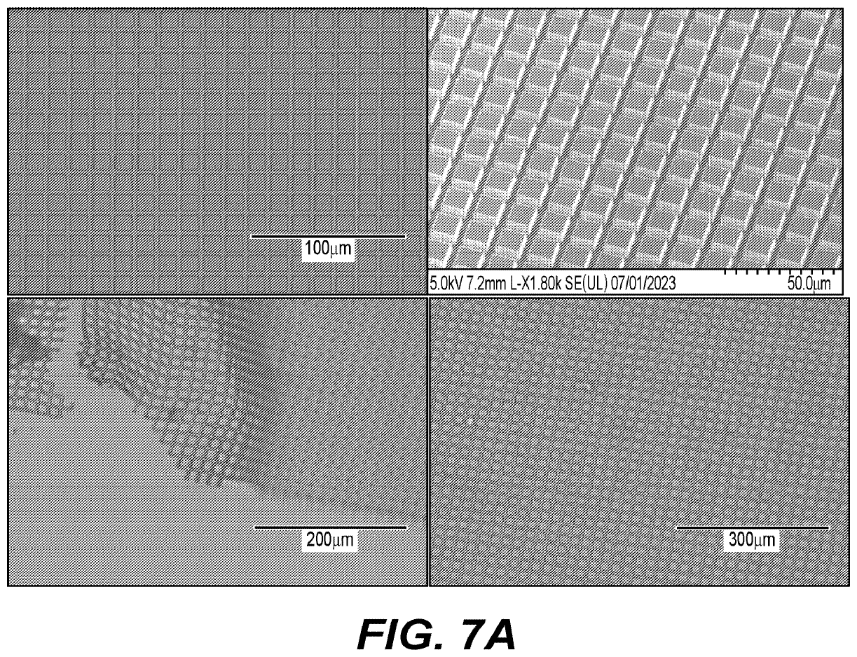

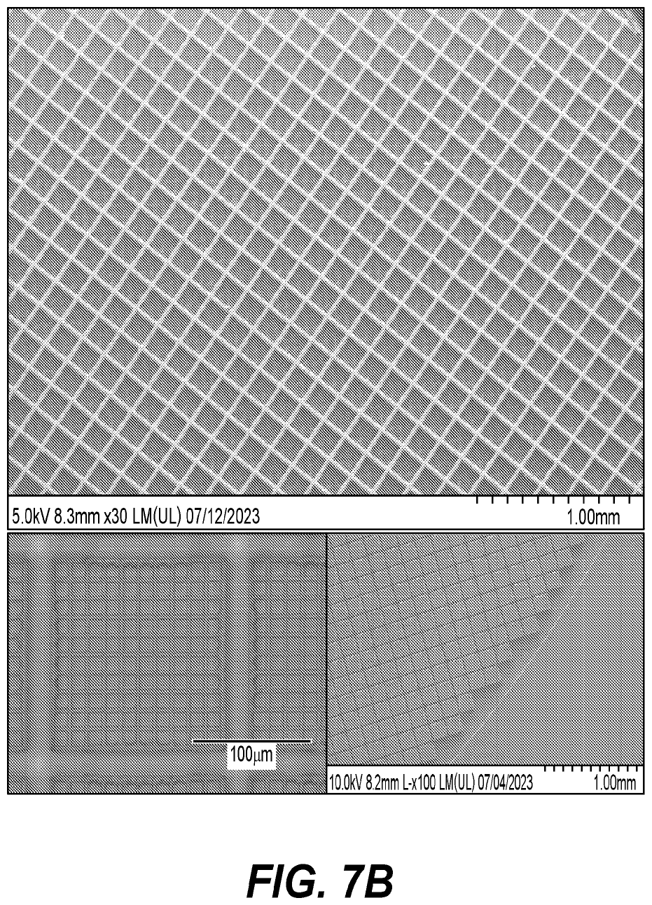

The extracorporeal circuit uses 5 main units (FIG. 9, FIG. 10A). A peristaltic pump is used for circulating the blood through the system which prevents strain on the animal's heart by aiding the flow. A 25 mm diameter version of the Cluster-Wells is used in a commercial filter holder for isolating the CTC clusters from the bloodstream. A passive bubble trap is employed for removing micro-bubbles that act as an embolus and may have an adverse effect on the animal. A heating unit with its own control circuit is used for keeping the blood temperature at physiological rates to prevent hypothermia in animals. Lastly, a flow and temperature sensors are utilized for measuring both volumetric flow rates and the temperature of blood returning to the animal.

Besides the main system components, the system employs 0.02″ ID tubing for transporting the blood in the system, an LCD screen module for showing the animal ID, flow rate, blood temperature and system operational time (FIG. 10B). Lastly, a printed circuit board (PCB) is designed for powering and controlling the individual system components, that include a microcontroller, a motor driver, 12V supply for heating elements, a power switch for wall adapter and a switch for powering the microcontroller (FIG. 10C).

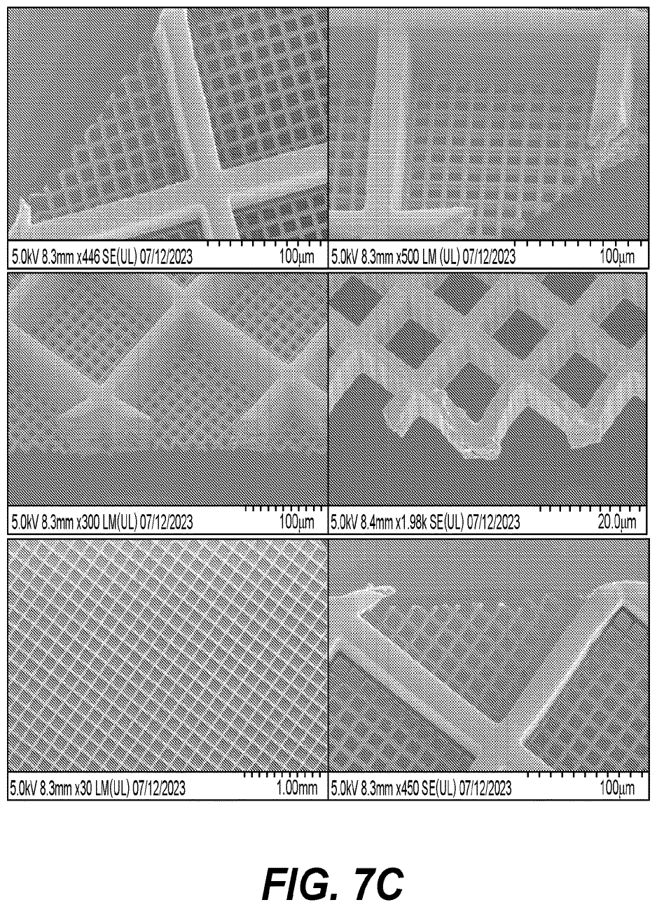

For homogeneous distribution of temperature within the system, a 3D printed casing was fabricated and filled it with thermally conductive beads which are heated by two heating elements. All components that carry processed blood were placed inside the casing for keeping the blood at the desired temperature throughout the experiments (FIG. 11). The dead-volume of the system is aimed to be within the blood sampling limits of rats to prevent further complications. The dead-volume of the finalized system was measured as ~2.3 mL, lower than the blood sampling limit of rats (2.5 mL).

One of the components of the extracorporeal system is the peristaltic pump which maintains the blood flow during operation. The peristaltic pump was chosen to have a stepper motor as the flow can be controlled much more precisely compared to a DC motor. The blood flow is dictated by the microcontroller by adjusting the step rate of the stepper motor. By considering the animal safety, the volumetric flow rate at the system initialization is set to 0.5 mL/min (300 motor step rate). The desired flow rate was achieved and maintained using a feedback control applied to the stepper motor which follows a transfer function. If the difference between the target and measured flow rates is lower than 1 mL/min, the motor step rate is changed by 10 for every interval (1 sec). Similarly, if the difference is more than 2 mL/h, the motor step rate is changed by 100. For the differences that fall between 1 mL/min to 2 mL/min, the rate of change is acquired from the transfer function. The motor step rate is changed until the measured flow rate is within ±5% tolerance of the target value, which maintains a constant flow rate throughout the experiment independently from the change in the fluidic resistance due to the captured clusters. Following the implementation of the feedback, we measured the flow stabilization times for various target flow rates. The study observed that the flow is stabilized in ~45 seconds for the target flow rate of 1 mL/min, ~50 seconds for 2 mL/min, ~55 seconds for 3 mL/min and ~68 seconds for 4 mL/min.

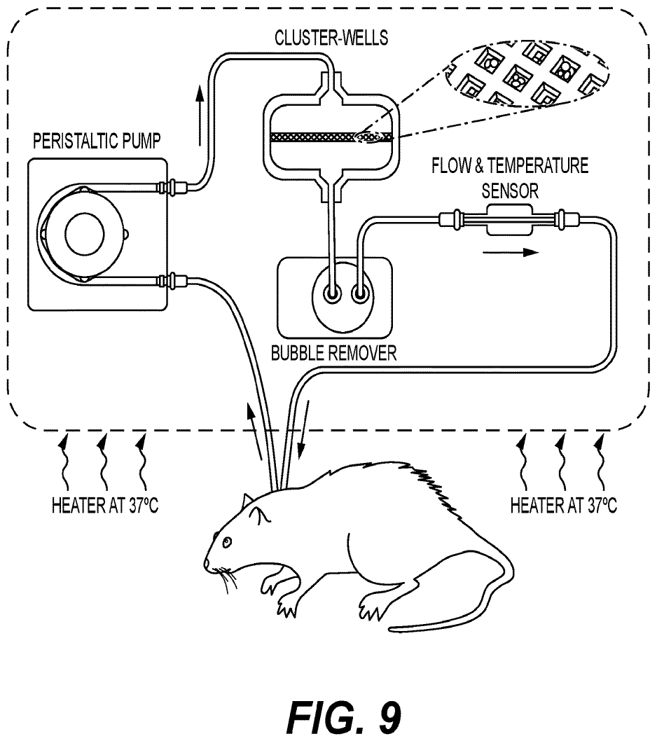

To assess the system's effect on the healthy blood cells, the study designed the experimental setup illustrated in FIG. 14. Blood samples donated from a healthy human subject was circulated through the extracorporeal system at 4 mL/min volumetric flow rate for 30 minutes, which corresponds to processing 120 mL of blood, ~5× of rat's total blood amount.

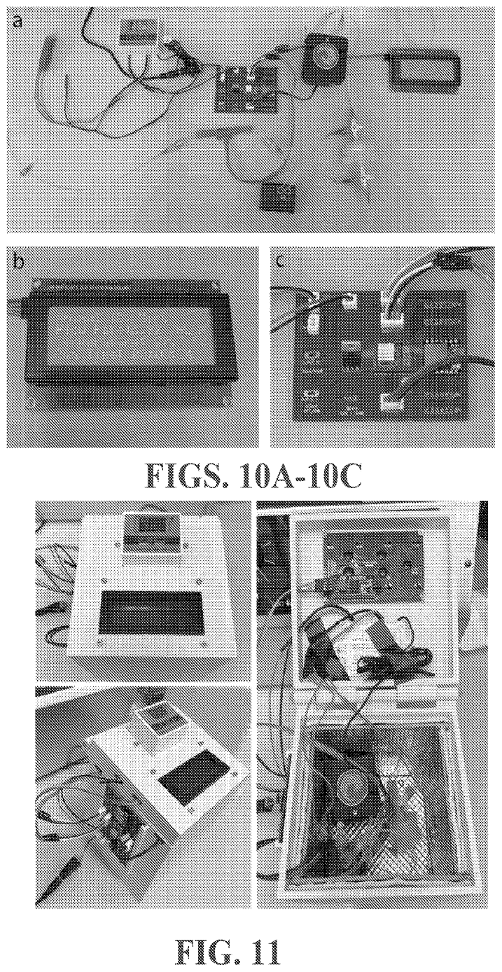

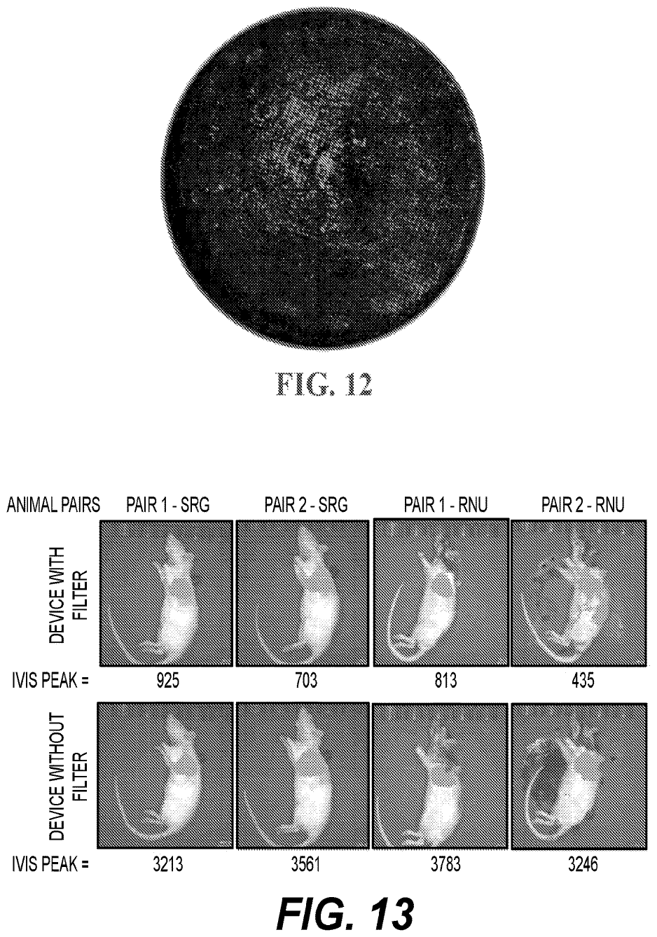

Following 30 minutes of system operation at 4 mL/min, we assessed the morphology of red blood cells and leukocytes (FIG. 15). To be able to observe individual cells under a microscope, the experiment diluted the samples 100 times using 1×PBS. To differentiate leukocytes from red blood cells, we performed live membrane staining for labeling the leukocytes using PE-CD45 (TRITC) conjugated antibody. When compared the processed population with the experimental control group, no morphological differences were observed between blood cells owing to the large size of the square pores that allows healthy cells to pass unimpededly. Lastly, the study subjected the leukocytes of the control and processed samples to a membrane integrity-based viability assay, NucRed Dead 647 Probes (Thermo Fisher). The cells were counted using a fluorescence microscope for determining the percentage of viable cells. The average viability of leukocytes in the processed sample was found to be similar to that of the control population (FIG. 16), showing the feasibility of the proposed workflow to be used with live animals.

### Retarding/Preventing Metastasis

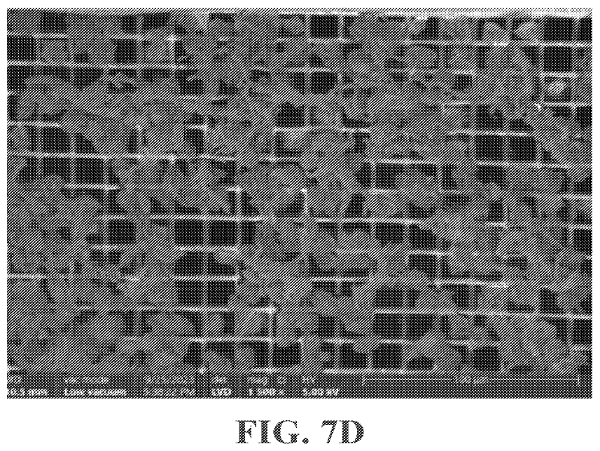

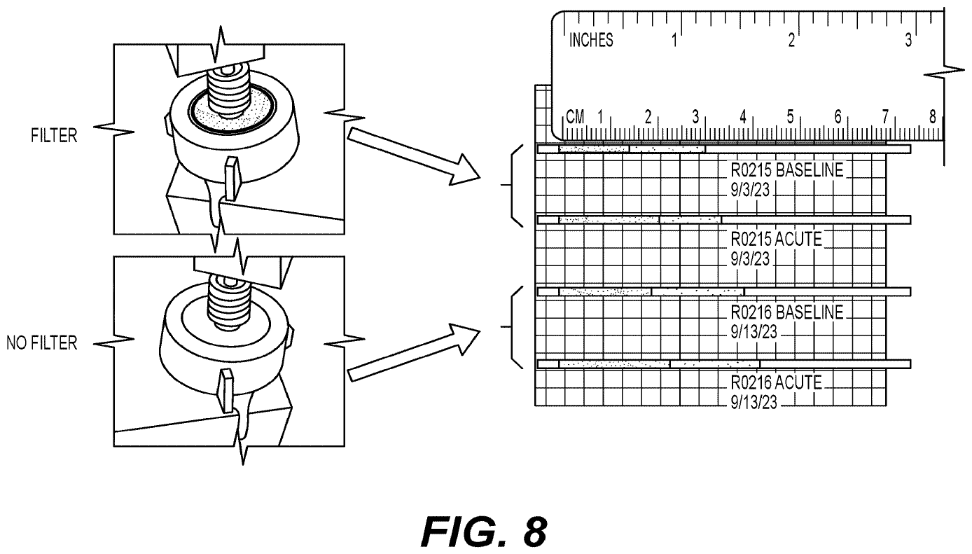

Besides isolation of large numbers of CTC clusters using the developed system, portable version can be operated continuously to remove CTC clusters from circulation. Being able to remove these metastatic precursors would significantly retard or potentially prevent metastasis of tumor. FIG. 12 and FIG. 13 show the effective capture of cell clusters using an example filter according to one aspect of the present disclosure.

### In-Line Cluster Dissociation

The designed system can be operated at higher shear forces which would dissociate the clustered cells into single cells during operation. Besides increasing shear force, any new cartridge (chip/device) specifically designed for dissociation purposes can be integrated into the system. Dissociating CTC clusters into single cells would decrease their metastatic propensity significantly. In some aspects, the high-flow filter assembly can be used for breaking apart clustered particles. For example, the volumetric flow rate in which a sample of blood is passed through the high-flow filter assembly can be increased such that the shear force on the captured clustered particles also is increased. The increase in shear force can cause dissociation of the clustered particles into non-clustered particles. In some examples, the size exclusion barrier comprises cluster-breaking elements forming a pointed tip to break apart clusters. By way of example, CTC clusters can be dissociated into single CTCs. Because single CTCs have been found to be less metastatic, this technique can facilitate therapeutic interventions and reduce instances of metastatic tumors developing.

Clot/Coagulation Detection and/or Dissociation

Apart from CTC clusters, clots, which are semi-solid masses circulating through blood, can also be detected and isolated from blood samples of patients/subjects. Similar to CTC clusters, high shear force or utilization of dissociation-specific devices can be used in the system to break down large masses into smaller pieces.

### Drug Delivery

The system can utilize its capture mechanism and integrate it with a personalized drug delivery for targeting tumor cell clusters captured on the chip. The system would allow the enrichment of CTC clusters and micro-dosing targeted cells locally instead of releasing large amounts of drug to the whole body. This technique would prevent side effects of drug-based cancer treatment.

### Thermal/UV-Based Cell Manipulation

The developed system can be used with external actuation mechanisms for manipulating isolated clusters. UV exposure could be coupled with our system for activating toxicity of antibody specific particles that targets cancer cells. Similarly, near infrared (NIR) light can be used for heating gold nanorods functionalized with tumor specific markers for specific heating of tumor cells. Localized heating would help to damage cancerous cells while not affecting healthy cells in the blood.

### Mechanical Cell Manipulation

Besides UV or thermal actuation, mechanical agitation can also be used for lysing captured clusters on the device. Longitudinal mechanical excitation using ultrasonic transducers would have an adverse effect on the isolated tumor cells and would end up lysing them while healthy cells that are exposed to mechanical excitation for a short period of time would not be affected.

Incorporation into Medical Devices

In some aspects, the system is operatively coupled to a medical device (e.g., implantable medical devices). In one example, the high-flow filter assembly can be operatively coupled to a stent. Because the high-flow filter assembly provides high In some aspects, the high-flow filter assembly can be positioned axially within a stent supporting a blood vessel where it is configured to prevent cell clusters from passing through the filter to the other side of the stent.

### Testing Kits

In some aspects, the system is included as a testing kit. In one example, the high-flow filter assembly is operatively coupled to a testing vessel (blood tubes, falcon tubes, -well and multi-well plates, cell strainer, etc.). In some aspects, a testing vessel having a bodily fluid and a filter can be placed in a centrifuge or gravity-based filter such that the bodily fluid moves through the filter assembly to separate cell clusters from the bodily fluid. Advantageously, the disclosed testing kits can be used as low-cost diagnostic tools in remote parts of the world.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications and publications as listed below and throughout this document are hereby incorporated by reference in their entirety herein.
M. Boya, T. Ozkaya-Ahmadov, B. E. Swain, C. H. Chu, N. Asmare, O. Civelekoglu, R. Liu, D. Lee, S. Tobia, S. Biliya, L. D. McDonald, B. Nazha, O. Kucuk, M. G. Sanda, B. B. Benigno, C. S. Moreno, M. A. Bilen, J. F. McDonald and A. F. Sarioglu, “High throughput, label-free isolation of circulating tumor cell clusters in meshed microwells”, Nature Communications, (2022).
T. Ozkaya-Ahmadov*, M. Boya*, Dohwan Lee, Mustafa Sak, Norh Asmare, Ozgun Civelekoglu, Can Firat Usanmaz, Sherry Tobia, L. DeEtte McDonald, Yujin Choi, Bassel Nazha, Benedict B. Benigno, John F. McDonald, Tobey J. MacDonald, Mehmet A. Bilen and A. Fatih Sarioglu, “Sacrificial biochips for isolation and unconstrained analysis of circulating tumor cells”, Science Advances (2022)—submitted.
O. Civelekoglu*, N. Wang*, A K M Arifuzzman, M. Boya and A. F. Sarioglu, “Automated lightless cytometry on a microchip with adaptive immunomagnetic manipulation”, Biosensors and Bioelectronics, (2022)
O. Civelekoglu, R. Liu, C. F. Usanmaz, C. H. Chu, M. Boya, T. Ozkaya-Ahmadov, A K M Arifuzzman, N. Wang and A. F. Sarioglu. “Electronic measurement of cell antigen expression in whole blood”, Lab on a Chip, (2021)
C. H. Chu, R. Liu, T. Ozkaya-Ahmadov, B. E. Swain, M. Boya, B. El-Reyes, M. Akce, M. A. Bilen, O. Kucuk and A. F. Sarioglu,
“Negative enrichment of circulating tumor cells from unmanipulated whole blood with a 3D printed device”, Scientific Reports, 11, 20583 (2021)
D. Lee, T. Ozkaya-Ahmadov, C. H. Chu, M. Boya, R. Liu and A. F. Sarioglu, “Capillary flow control in lateral flow assays via delaminating timers”, Science Advances, 7, 40(2021)
C. H. Chu, R. Liu, T. Ozkaya-Ahmadov, M. Boya, B. E. Swain, J. M. Owens, E. Burentugs, M. A. Bilen, J. F. McDonald and A. F. Sarioglu, “Hybrid negative enrichment of circulating tumor cells from whole blood in a 3D-printed monolithic device”, Lab on a Chip, 19, 3427 (2019)
O. Civelekoglu, N. Wang, M. Boya, T. Ozkaya-Ahmadov, R. Liu and A. F. Sarioglu, “Electronic profiling of membrane antigen expression via immunomagnetic cell manipulation”, Lab on a Chip, 19, 2444 (2019)
R. Liu, W. Waheed, N. Wang, O. Civelekoglu, M. Boya, C. H. Chu and A. F. Sarioglu, “Design and modeling of electrode networks for code-division multiplexed resistive pulse sensing in microfluidic devices”, Lab on a Chip, 17, 2650 (2017)
M. Boya, A. Fatih Sarioglu, “Cluster-Wells: A technology for routine and rapid isolation of extremely rare circulating tumor cells clusters from unprocessed whole blood”, Chapter in “Microfluidic Systems for Cancer Diagnosis”, Springer Science+Business Media (2022)—in press
M. Boya, C. H. Chu, R. Liu, T. Ozkaya-Ahmadov and A. F. Sarioglu, “Circulating Tumor Cell Enrichment Technologies”,
Chapter 2 of Tumor Liquid Biopsies, pp. 25-55, edited by F. Schaffner, J-L. Merlin, N. von Bubnoff, Springer Nature, (2020).
U.S. Patent Publication No. 2022/0298489 A1

1. A system for the in-line removal of cell clusters from a bodily fluid (e.g., blood sample) of a subject, the system comprising:
a high-flow filter assembly having a filter substrate that forms a plurality of filtering elements (e.g., semipermeable microwells), the filter assembly being configured to receive (e.g., directly) an inlet fluid (e.g., unfiltered blood) from a subject at a first side and output a filtered output at a second side, wherein each filtering element defines a plurality of apertures extending between the first side and the second side of the filter, each filtering element forming a cluster-exclusion barrier (e.g., a size-exclusion barrier) in a trapping region of the filtering element to substantially prevent cell clusters of multiple cells (e.g., circulating tumor cell (CTC) clusters) from passing from the first side to the second side.

2. The system of claim 1, further comprising a pump configured to move the inlet fluid towards or from the high-flow filter assembly.

3. The system of claim 1, wherein the size-exclusion barrier comprises an interconnected mesh defining a plurality of spaced openings and/or cluster-breaking elements configured to at least partially separate cell clusters.

4. (canceled)

5. The system of claim 1, further comprising a bubble remover in fluid communication with the high-flow filter assembly, wherein the bubble remover is configured to remove gases from the filtered output.

6. The system of claim 5, wherein the bubble remover comprises an inlet stream of the filtered output entering a housing of the bubble remover, wherein the inlet stream is configured to contact a substantially immiscible liquid having a lower density than the filtered output.

7. The system of claim 6, wherein the inlet stream is configured to enter the bubble remover substantially towards a side wall of the housing or substantially away from a side wall of the housing.

8. (canceled)

9. The system of claim 1, further comprising a heating unit configured to supply thermal energy to the fluid inlet and/or the filtered output.

10. The system of claim 1, wherein the filter substrate of the high-flow filter assembly is coupled to a filter holder.

11. The system of claim 10, wherein the filter holder comprises a sealing region, wherein the sealing region defines an area enclosing at least a portion of the high-flow filter assembly, wherein the sealing region is configured to substantially prevent the inlet fluid from passing through the filter substrate outside of the area.

12. The system of claim 11, wherein the sealing region is substantially non-porous to the inlet fluid.

13. The system of claim 1, wherein the filter holder is integrally formed on the filter substrate.

14. The system of claim 1, wherein the inlet fluid is configured to contact a surface of the filter substrate at a contact angle, wherein the contact angle is normal to the surface.

15. (canceled)

16. (canceled)

17. (canceled)

18. (canceled)

19. The system of claim 1, wherein the high-flow filter assembly has a three-dimensional shape.

20. The system of claim 1, wherein the filter substrate comprises a biocompatible material, a UV curable polymer, a printable material, and/or a disposable material.

21. (canceled)

22. (canceled)

23. (canceled)

24. The system of claim 1, wherein the system is a closed-loop system.

25. The system of claim 1, further comprising sensors.

26. The system of claim 1, further comprising an imaging unit configured to image and/or screen retained cell clusters.

27. (canceled)

28. The system of claim 1, further comprising a plurality of high-flow filter assemblies arranged in parallel fluid circuits or sequential fluid circuits.

29. (canceled)

30. The system of claim 1, wherein the high-flow filter assembly is operatively coupled to an implantable device or a stent.

31. (canceled)

32. A method for the in-line removal of cell clusters, the method comprising:
causing a volume of a fluid of a subject to circulate within the in-line filtration system according to claim 1.

33-42. (canceled)
